(19) United States
(12) Patent Application Publication
(10) Pub. No.: US 2009/0088471 A1
(43) Pub. Date: Apr. 2, 2009

(54) Chemical sterilant for adult male dog population control with concomitant reduction in human dog-bite acquired rabies

(76) Inventor: Min Wang, Columbia, MO (US)

Correspondence Address: GRACE J FISHEL, 2200 WEST PORT PLAZA DRIVE, SUITE 202, ST. LOUIS, MO, 63146, US

(21) Appl. No.: 11/863,758

(22) Filed: Sep. 28, 2007

(51) Int. Cl. A61K 31/315

(52) U.S. Cl. 514/494

(57) ABSTRACT

A chemical composition for use in controlling the population of free-roaming, sexually mature male dogs. The chemical sterilant is injected in a manner that the dog's appearance and secondary male sexual characteristics are preserved such that the dog maintains its position in the hierarchy of the free-roaming dogs while not contributing to the number of puppies produced. A reduction in the number of free-roaming dogs reduces the number of human victims of dog-bite acquired rabies. Because the appearance and nature of the sterilized dog is substantially unchanged, the chemical composition is acceptable to the human community that the dog habitats.

### BACKGROUND OF THE INVENTION

1. Field of the Invention

The present invention relates to an aqueous solution of a mineral gluconate salt and an amino acid capable of forming the solution neutralized to a pH in the range of 6.0 to 7.5 for use as a field-administered chemical sterilant for population control of free-roaming, sexually mature male dogs.

2. Brief Description of the Prior Art

Dog bites are the cause of the vast majority of human rabies cases in developing countries wherein dogs roam freely throughout the human community and no one takes responsibility for controlling their reproduction. It is generally believed that free-roaming dogs exist in very few places where they are strictly feral and have no referral household or community (WHO 1990). Many free-roaming dogs are so called neighborhood dogs which are semidependent on one or more families for food and shelter. Neighborhood dogs range within a generally defined area and are tolerated in the community perhaps because they keep populations of other less desirable creatures such as rats and mice under control. Neighborhood dogs may also provide protection against other animals and human interlopers. Other free-roaming dogs are family dogs which are essentially wholly dependent on one family for food but are allowed to roam part of the time.

In many developing countries, efforts to control the number of free-roaming dogs have focused on killing them, often using inhumane methods such as poisoning, mass electrocution, beating or drowning. In many cases these campaigns do not discriminate between the owned-but-roaming animals and neighborhood dogs in an area. Consequently, there is often considerable antagonism between the government functionaires charged with collecting dogs and the population at large. In addition, dog removal may indeed be counterproductive when considered from a rabies-control perspective. In the presence of an empty biological niche with unexploited resources, more puppies are born to the surviving animals and more of them survive. More dogs also migrate into the area recently rendered dog-free to the end that the area is again quickly overpopulated.

In reaction to the inhumane and ultimately unsuccessful control of free-roaming dog populations by killing, animal welfare groups have promoted Animal Birth Control (ABC) programs. The basic premise behind ABC programs is that captured dogs are sterilized, vaccinated against rabies, and returned to the exact location where they were caught. The sterilized dogs thus maintain their position in the hierarchy of free-roaming dogs, preventing migration while not contributing to the number of puppies produced.

Surgical sterilization is the most common method but this requires that the animal be prepared individually for surgery and given anesthetic, antibiotics and analgesics. Finding funds, infrastructure to capture and release the dogs and veterinary expertise necessary are difficult or impossible. More male dogs are surgically sterilized than female dogs because castration is an easier surgical procedure than ovariohysterectomy. However, a surgically castrated male dog loses some of his secondary male characteristics, such as aggressiveness, which makes him less desirable as a family or neighborhood watchdog. Loss of aggressiveness may also affect the dog's standing in the hierarchy of free-roaming dogs and contribute to population instability.

As an alternative to knife castration, an injectable sterilization compound for male dogs would be a great advance because it would:

(1) Be less invasive and less painful for the dog;
(2) Be easier and quicker to administer than castration with a knife;
(3) Likely be done without transporting the animal to a central veterinary clinic;
(4) Allow more dogs to be sterilized in the same time period and at a much lower cost in transport, kenneling and feeding costs in preparation for and recovery from surgery; and,
(5) Provide a means of sterilizing dogs in areas where veterinary resources are limited.

As mentioned above, however, the human community has an emotional investment in the free-roaming dogs. Hence to be acceptable to the community (i.e., the functionaries not met with resistance), effectiveness in sterilization is not enough. The treatment must also not change the physical appearance or demeanor of the treated male dogs significantly to be acceptable to the referral household or community.

The present invention provides an injectable sterilization compound which satisfies the above-mentioned requirements. The sterilized male dogs continue to occupy the biological niche in which they were found and prevent the migration of fertile male dogs. Fewer puppies are born and the number of free-roaming dogs declines. With fewer dogs, there are fewer dog-bites and the incidence of human rabies declines.

### BRIEF SUMMARY OF THE INVENTION

In view of the above, it is an object of the present invention to provide a chemical sterilant for population control of free-roaming, sexually mature male dogs. It is another object to provide a chemical sterilant that reduces the population of free-roaming dogs, which, in turn, reduces human dog-bite acquired rabies. Other objects and features of the invention will be in part apparent and in part pointed out hereinafter.

In accordance with the invention, a chemical sterilant for adult male dog population control is provided. The chemical sterilant is an aqueous solution of a mineral gluconate salt and an amino acid capable of forming the solution. The aqueous solution is neutralized to a pH in the range of 6.0 to 7.5 for injection into a free-roaming, sexually mature dog that occupies a position in a hierarchy of free-roaming dogs in a biological niche. The sexually mature dog has scrotal testes with seminiferous tubules flowably connected to a head of an epididymis by tubuli recti, rete testis and ductuli efferentes. The sexually mature dog is captured for sterilization in the biological niche, the sterilant injected into a dorsal cranial portion of each testis beside the head of the epididymis in an effective amount to achieve sterilization without substantially affecting the dog's male physical appearance or secondary sexual characteristics and the sexually mature dog is immediately released back into the biological niche without upsetting the hierarchy.

The invention summarized above comprises the compositions hereinafter described, the scope of the invention being indicated by the subjoined claims.

### BRIEF DESCRIPTION OF THE SEVERAL VIEWS OF THE DRAWING

In the accompanying drawings, FIG. 1 is a histology of a puppy testis characterized by

small diameter of seminiferous tubules
seminiferous tubules mainly composed of Sertoli cells and spermatogonia
relatively large interstitial tissue with a few Leydig cells;

FIG. 2 is a histology of a sexually mature dog testis characterized by

large diameter of seminiferous tubules
seminiferous tubules filled with each type of germinal cells and with active spermatogenesis
large number of Leydig cells in the interstitial tissue; and,

FIG. 3 is a schematic representation of a sexually mature dog testis with an arrow indicating an injection site for the chemical sterilant.

### DETAILED DESCRIPTION OF THE INVENTION

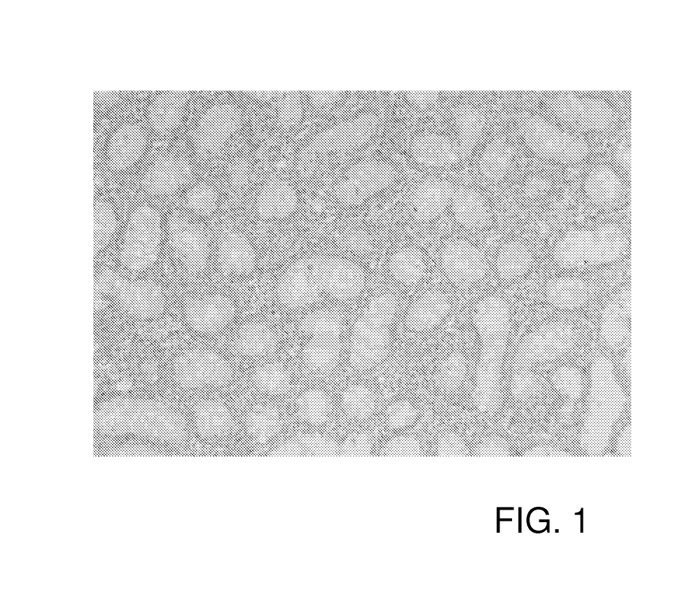

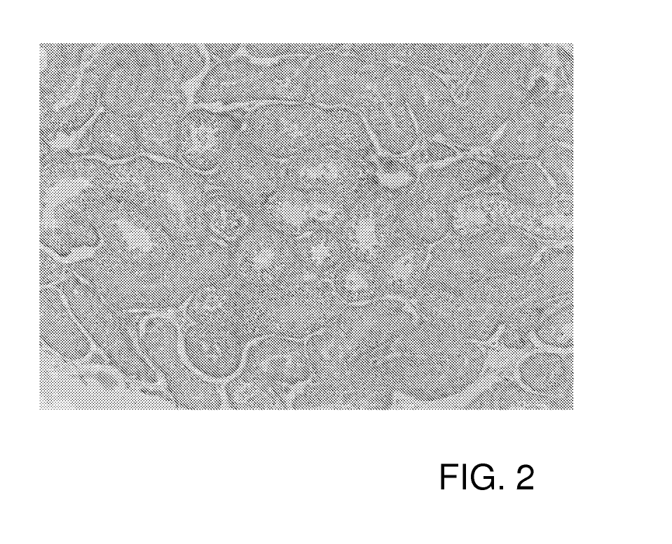

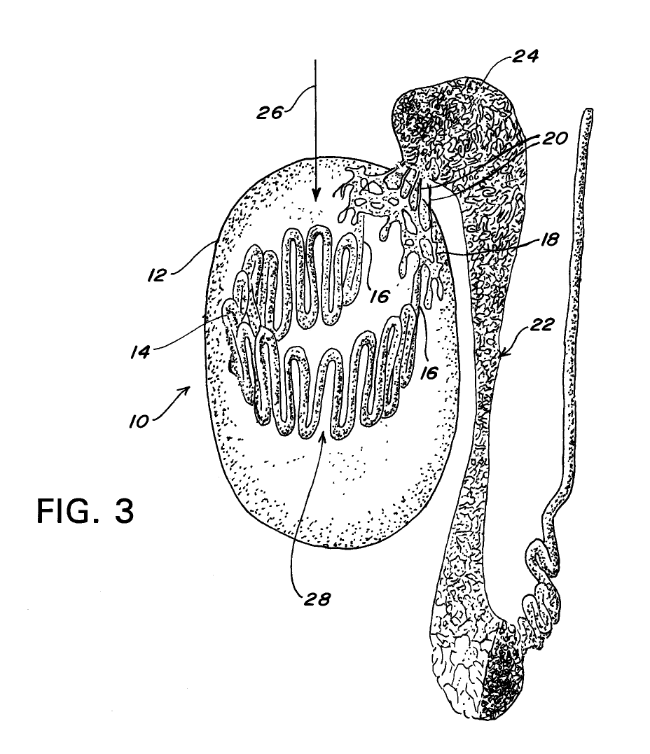

Referring to the drawings more particularly by reference character, a sexually mature, free-roaming male dog having scrotal testes, a histology of which is shown in FIG. 2 and a cross-section of which is shown schematically in FIG. 3, is captured in the field for sterilization with a chemical sterilant. Prior to capture, the sexually mature dog occupies a position in a hierarchy of free-roaming dogs in a biological niche. The chemical sterilant, as more particularly described below, effects sterilization without significantly affecting the dog's physical appearance or secondary sexual characteristics. When the sterilized dog is returned back into the biological niche where it was captured, it maintains its position in the hierarchy of free-roaming dogs, preventing migration of fertile male dogs while not contributing to the number of puppies produced. As the population of free-roaming dogs declines, the incidence of human dog-bite acquired rabies also declines. This impact is enhanced if the male dogs are also immunized against rabies at the time they are sterilized. Funds for this purpose may be available since the neutered dogs need not be transported to a central veterinary clinic for treatment.

The chemical sterilant is a mineral gluconate salt and an amino acid capable of forming an aqueous solution neutralized to a pH in the range of 6.0 to 7.5. Physiologically acceptable minerals include zinc, calcium, iron, magnesium, manganese and the like and illustrative mineral salts include zinc gluconate.

Zinc gluconate can be neutralized to form a stable aqueous solution with the following amino acids: alanine, valine, isoleucine, proline, glycine, serine, threonine, asparagine, glutamine, lysine, arginine, histidine and mixtures thereof. The solution cannot be formed with cysteine, tyrosine, aspartic acid or glutamic acid and among the basic amino acids, arginine is preferred when the mineral gluconate salt is zinc gluconate.

In neutralizing mineral salts such as zinc gluconate, it is preferred that the mineral salts and the amino acid be present in substantially equimolar amounts. Suitable formulations for use as a chemical sterilant are formed with a molar amount of mineral salt such as zinc gluconate to amino acid such as arginine from about 0.05 M:2.0 M to about 2.0 M:0.05 M, preferably from about 0.05 M:0.3 M to about 0.3 M:0.05 M and most preferably from about 0.1 M:0.2 M to about 0.2 M:0.1 M and neutralized to a pH in the range from about 6.0 to about 8.0, preferably from about 6.5 to about 7.5 and most preferably 7.0. The solution is formed and then sterile filtered into sterile rubber-stoppered glass vials.

It is desirable to inject the lowest possible effective amount of chemical sterilant into each testis (see FIG. 3) of the sexually mature, free-roaming dogs captured for sterilization. As shown in FIG. 3, testis 10 has an oval structure with an outer covering, the fibrous tunica albuginea 12 thickened posteriorly along the epididymal border, where it forms the mediastinum. The tunica albuginea 12 is composed of three layers: an outer layer called the tunica vaginalis, a middle layer called the tunica albuginea proper, and inner layer called the tunica vasculosa which is a subtunical extension of the interstitial tissue 28 consisting of blood vessels and some Leydig cells in a loose connective tissue. The Leydig cells synthesize and secrete testosterone which is required to maintain spermatogenesis and male secondary sexual characteristics. The interstitial tissue divides each testis into compartments enclosing one or more seminiferous tubules 14. Sperm are produced in the seminiferous tubules 14.

Each seminiferous tubule 14 is lined on its inside by the seminiferous epithelium, which contains two kinds of cells—male germ cells and Sertoli cells. Sperm develop in the seminiferous tubules 14 from less mature cell types. The least mature germinal cells, the spermatogonia, divide to form primary spermatocytes. The primary spermatocytes divide meiotically to form secondary spermatocytes which, in turn, divide mitotically to form spermatids. Spermatids do not divide further, but undergo a complicated metamorphosis in the process of forming sperm. The Sertoli cells nurture the spermatids and secrete a fluid that washes the sperm along the seminiferous tubules 14.

The seminiferous tubules 14 form two-ended, convoluted loops, the two terminal portions of which connect with the tubuli recti 16. Sperm, suspended in testicular fluid, leave the seminiferous tubules and enter the tubuli recti 16. These tubules, in turn, join the rete testis 18 which is a network of tubules within the testis 10. At the upper end of the mediastinum, the vessels of the rete testis 18 terminate in the ductuli efferentes 20 which pass through the tunica albuginea 12 and carry the seminal fluid containing sperm from the testis to the epididymis 22 where sperm further mature and are stored. The passage of sperm through the seminiferous tubules 14, tubuli recti 16, rete testis 18 and ductuli efferentes 20 is passive. The cells lining these ducts have cilia and the beating of these hairlike structures move the fluid and the suspended sperm through the ducts and into the head of the epididymis 24.

The sperm produced in the seminiferous tubules 14 must undergo a series of changes before they are capable of fertilizing an egg. The journey starts with safe passage through the tubuli recti 16, rete testis 18, ductuli efferentes 20 into the head of the epididymis 24. The epithelium of the tubuli recti 16 and rete testis 18 add fluids which are then reabsorbed by the epithelium in the ductuli efferentes 20. The composition of the fluids in the tubuli recti 16, rete testis 18 and ductuli efferentes 20 is regulated and essential to the provision of viable cells to the epididymis 22 for further processing into mature sperm.

When the chemical sterilant is injected from the side or bottom of testis 10, occasionally a small portion of the testis is left intact after treatment. Increasing the dose above the amount expected to be effective is not desirable but injecting the chemical sterilant into the dorsal cranial portion of the testis beside the epididymis 22 (i.e., in the direction of arrow 26 as shown in FIG. 3) effects complete sterilization in substantially all cases with minimal dose.

The injection into the dorsal cranial portion has an effect on the epithelium of the tubuli recti 16, rete testis 18 and ductus efferentes 20 in addition to the seminiferous tubules 14. If some of the seminiferous tubules 14 in a portion of the testis 10 remain intact, any sperm produced must pass through the above-mentioned transportation system to have any chance at maturing in the epididymis 22 into sperm capable of fertilizing an egg. By affecting the epithelium of the tubuli recti 16, rete testis 18 or ductus efferentes 20, the tubes may not add and remove fluids as is required for the successful development or maintenance of the sperm and there may be no cilia to sweep them along. Hence even if produced in some portion of the testis 10, no viable sperm reach the epididymis 22 so that the sterilization is complete. The Leydig cells, however, continue to produce testosterone in an amount sufficient to maintain the dog's physical appearance and secondary sexual characteristics, such as male aggressiveness. This allows the animal to maintain its social position in the biological niche it occupied prior to treatment.

The following examples illustrate the invention.

### EXAMPLE 1

Zinc gluconate neutralized by arginine injectable solution has been approved by the Food and Drug Administration (FDA) for intratesticular injection in puppies, 3 to 10 months of age. (Freedom of Information Summary, NADA, 141-217, United States Food and Drug Administration, Mar. 17, 2003). The clinical study in puppies showed it was safe and effective for chemical sterilization of sexually immature dogs when used under label conditions.

A comparison of FIGS. 1 and 2 shows that the histology of a puppy testis and a sexually mature dog testis differ significantly. The safety and effectiveness of zinc gluconate neutralized with arginine had not been documented in sexually mature dogs and, therefore, the purpose of this example was to obtain safety and efficacy data from sexually mature, male dogs older than 10 months of age, using the same dose and administration as approved by the FDA for use in 3 to 10 month old puppies.

The test substance was a sterile injectable aqueous solution containing 0.2 M zinc gluconate neutralized to pH 7.0 with 0.2 M L-arginine (13.1 mg zinc per mL). The test substance was provided in sterile rubber-stoppered glass vials containing 2 mL of solution. The solution contained no preservatives; therefore, each vial was used to treat a single dog.

Dosage was based upon testicular width according to Table 1 below:

TABLE I

| Range of Testicular Width | Dose Per Testis |
|---|---|
| (mm) | mL |
| 10-12 | 0.2 |
| 13-15 | 0.3 |
| 16-18 | 0.5 |
| 19-21 | 0.7 |
| 22-24 | 0.8 |
| 25-27 | 1.0 |

Treatment was performed according to the following procedure:
1. The necessity for chemical restraint was determined. Chemical restraint was usually not necessary but investigators had the option of using chemical restraint at their discretion. If chemical restraint was used, acepromazine, ketamine or similar approved sedative/tranquilizer was used.
2. The width of each testis was measured at the widest part using a specifically designed dosage caliper as described in U.S. Pat. No. 7,125,385.
3. Gross dirt and debris were removed from the scrotum with a mild surgical soap such as chlorhexidine diacetate diluted 1:10.
4. A 1 cc syringe with 25 gauge, 1 inch needle was used to inject the drug. A separate sterile needle was used for each testis since using the same needle for the two testes might have led to infection.
5. The zinc gluconate neutralized by arginine was injected at the dorsal cranial portion of the testis beside the caput (head) of the epididymis so that the drug not only diffused into the rete testis but also into the caput of the epididymis as shown in FIG. 3. This placement of the injection prevented maturation of sperm and caused atrophy of the ductuli efferentes and rete testis and prevented the passage of sperm to the caput of the epididymis, thereby achieving sterilization more effectively.
6. The injection technique was as follows:
   a. The needle (25 gauge, 1 inch) was inserted into the dorsal cranial portion of each testis (FIG. 3).
   b. Care was taken not to inject the drug into the scrotum or intradermally into the scrotal skin. When the injection was made, the skin of the scrotum was held tight over the testis to avoid loose scrotal tissue over the testis that would lead to injection into the scrotum.
   c. The drug was injected slowly because rapid injection might have stimulated contraction of the seminiferous tubules and the drug might have leaked from the injection site.
   d. Excessive injection pressure was avoided and if resistance was felt, injection was discontinued.
7. Since there is no currently approved chemical sterilant available for use in sexually mature, male dogs, the drug's effectiveness was compared to a historical control by comparing the results of semen analyses to the known analysis of a normal, healthy, intact male dog.

Fifty-four sexually mature, male dogs of any breed and older than 10 months of age were selected for the study. Each dog underwent physical examination with the following selection parameters:

All dogs included in the study were:
a. Sexually mature (older than 10 months of age) and had normal semen parameters;
b. Had no abnormalities on physical examination;
c. Had been immunized against distemper, hepatitis, leptospirosis, para influenza, parvo (DHLPP); and rabies.

Criteria for exclusion from entry into the investigation were:

a. Testicular width below 10 mm or above 27 mm.

b. Undescended testicle or testicles;

c. Ulceration and/or cuts on the scrotum;

d. Fibrosis in testes or epididymides.

Each of the fifty-four sexually mature, male dogs selected for the study was assigned a unique identification number by using a tattoo or microchip animal identification. The dogs were retained by their owners and visited by veterinarians from the Public Health Department and Veterinary College of the Universidad Nacional Autonoma de Mexico (UNAM) for observation during the investigational period. The veterinarians visited every 24 hours during the first week post-injection and then at two month, four month and six months post injection. The body weight of the dogs is reported in Table II below.

TABLE II

| Age | | | | Post-Injection | | | Individual Dog | |
|---|---|---|---|---|---|---|---|---|
| (years) | Dog No | Dog Name | Day 0 | 2 Months | 4 Months | 6 Months | Average | S.D. |
| 1 | 01RP06 | Dutch | 25.0 | 25.0 | 25.0 | 25.0 | 25.0 | 0.0 |
| 1 | 09RP05 | Yogui | 14.0 | 14.0 | 14.0 | 14.0 | 14.0 | 0.0 |
| 1 | 14RH01 | Solovino | 15.0 | 15.0 | 15.0 | 15.0 | 15.0 | 0.0 |
| 1.5 | 17RM05 | Sultan | 15.0 | 15.0 | 15.0 | 15.0 | 15.0 | 0.0 |
| 1.5 | 29RP05 | Chicles | 7.0 | 7.0 | 7.0 | 7.0 | 7.0 | 0.0 |
| 2 | 01RP05 | Pompin | 10.0 | 10.0 | 10.0 | 10.0 | 10.0 | 0.0 |
| 2 | 03RP05 | Dexter | 30.0 | 30.0 | 30.0 | 30.0 | 30.0 | 0.0 |
| 2 | 06RP05 | Bobby II | 19.0 | 19.0 | 19.0 | 19.0 | 19.0 | 0.0 |
| 2 | 10RM05 | Firulais | 25.0 | 25.0 | 25.0 | 25.0 | 25.0 | 0.0 |
| 2 | 10RM06 | Caruzo | 24.5 | 25.0 | 25.0 | 25.0 | 24.9 | 0.3 |
| 2 | 11RM06 | Gordo | 23.0 | 25.0 | 25.0 | 26.0 | 24.8 | 1.3 |
| 2 | 11RP05 | Yanki | 25.0 | 25.0 | 25.0 | 25.0 | 25.0 | 0.0 |
| 2 | 13RP05A | Rocky | 18.0 | 18.0 | 18.0 | 18.0 | 18.0 | 0.0 |
| 2 | 13RP05B | Roger | 20.0 | 20.0 | 20.0 | 20.0 | 20.0 | 0.0 |
| 2 | 15RM05 | Manchas | 18.0 | 18.0 | 20.0 | 18.0 | 18.5 | 1.0 |
| 2 | 15RP05 | Duque | 20.0 | 20.0 | 20.0 | 20.0 | 20.0 | 0.0 |
| 2 | 18RP05 | Boby | 12.0 | 12.0 | 12.0 | 12.0 | 12.0 | 0.0 |
| 2 | 19RM05 | Wester | 22.0 | 22.0 | 22.0 | 22.0 | 22.0 | 0.0 |
| 2 | 30RP09 | Puppy | 13.0 | 13.0 | 13.0 | 13.0 | 13.0 | 0.0 |
| 2 | 3RM05 | Killer | 17.0 | 17.0 | 17.0 | 17.0 | 17.0 | 0.0 |
| 2 | 4RM05 | Rucky | 18.0 | 18.0 | 18.0 | 18.0 | 18.0 | 0.0 |
| 2 | 4RP06 | Obelix | 27.0 | 27.0 | 27.0 | 27.0 | 27.0 | 0.0 |
| 2 | 7RM05 | Ramses | 20.0 | 20.0 | 20.0 | 20.0 | 20.0 | 0.0 |
| 2 | 7RP06 | Ramiro | 27.0 | 27.0 | 27.0 | 27.0 | 27.0 | 0.0 |
| 2.5 | 1RM06 | Hugo | 23.0 | 28.0 | 28.0 | 28.0 | 26.8 | 2.5 |
| 3 | 10RH01 | Oso | 15.0 | 15.0 | 15.0 | 15.0 | 15.0 | 0.0 |
| 3 | 10RP09 | Peluchin | 11.0 | 11.0 | 11.0 | 11.0 | 11.0 | 0.0 |
| 3 | 14RP09 | Terri | 22.0 | 22.0 | 22.0 | 22.0 | 22.0 | 0.0 |
| 3 | 15RP09 | Pinto | 20.0 | 20.0 | 20.0 | 20.0 | 20.0 | 0.0 |
| 3 | 1RH01 | Chacho | 12.0 | 12.0 | 12.0 | 12.0 | 12.0 | 0.0 |
| 3 | 20RP09 | Tribilin | 17.0 | 17.0 | 17.0 | 17.0 | 17.0 | 0.0 |
| 3 | 22RM05 | Beethoven | 23.0 | 23.0 | 23.0 | 23.0 | 23.0 | 0.0 |
| 3 | 2RM06 | Brandy | 27.0 | 30.0 | 30.0 | 30.0 | 29.3 | 1.5 |
| 3 | 31RP05 | Flay | 11.0 | 11.0 | 11.0 | 11.0 | 11.0 | 0.0 |
| 3 | 36RP09 | Duque | 19.0 | 19.0 | 19.0 | 19.0 | 19.0 | 0.0 |
| 3 | 8RM06 | Bloky | 27.0 | 27.0 | 27.0 | 27.0 | 27.0 | 0.0 |
| 3 | 9RM05 | Bigotes | 14.0 | 14.0 | 14.0 | 14.0 | 14.0 | 0.0 |
| 4 | 1RH04 | Aragorn | 12.0 | * | * | * | 12.0 | * |
| 4 | 26RP05 | Bando | 17.0 | 17.0 | 17.0 | 17.0 | 17.0 | 0.0 |
| 5 | 11RH01 | Abelardo | 19.0 | 19.0 | 19.0 | 19.0 | 19.0 | 0.0 |
| 5 | 19RH01 | Tejon | 18.0 | 18.0 | 18.0 | 18.0 | 18.0 | 0.0 |
| 5 | 2RM05 | Bobby | 20.0 | 20.0 | 20.0 | 20.0 | 20.0 | 0.0 |
| 5 | 32RP05 | Jordi | 23.0 | 23.0 | 23.0 | 23.0 | 23.0 | 0.0 |
| 5 | 5RM06 | Peluchin | 7.0 | 7.0 | 7.0 | 7.0 | 7.0 | 0.0 |
| 5 | 9RM06 | Muneco | 17.0 | 20.0 | 20.0 | 20.0 | 19.3 | 1.5 |
| 6 | 11RM05 | Scooby | 18.0 | 18.0 | 18.0 | 18.0 | 18.0 | 0.0 |
| 6 | 13RM05 | Chiquilin I | 17.0 | 17.0 | 17.0 | 17.0 | 17.0 | 0.0 |
| 6 | 14RM05 | Chiquilin II | 25.0 | 25.0 | 25.0 | 25.0 | 25.0 | 0.0 |
| 6 | 5RM05 | Bombon | 18.0 | 18.0 | 18.0 | 18.0 | 18.0 | 0.0 |
| 7 | 13RH01 | Max | 25.0 | 25.0 | 25.0 | 25.0 | 25.0 | 0.0 |
| 7 | 20RP05 | Golfo | 20.0 | 20.0 | 20.0 | 20.0 | 20.0 | 0.0 |
| 8 | 18RH01 | Tulipan | 17.0 | 17.0 | 17.0 | 17.0 | 17.0 | 0.0 |
| 12 | 16RM05 | Blanco | 19.0 | 17.0 | 17.0 | 19.0 | 18.0 | 1.2 |
| 12 | 6RH01 | Peluche | 5.0 | 6.0 | 6.0 | 7.0 | 6.0 | 0.8 |

| | | | | | |
|---|---|---|---|---|---|
| Average | (54 dogs) | 18.4 | 18.8 | 18.8 | 18.9 |
| S.D. | (54 dogs) | 5.6 | 5.8 | 5.8 | 5.8 |

* castrated

### EXAMPLE 2

When the dogs were examined and weighed as described in Example 1, the testicular width of each testis was also measured. Results for right testicular widths and percentage change from day 0 are shown in Table III and for left testicular widths are shown in Table IV below.

TABLE III

| | | | | 2 Months | | 4 Months | | 6 Months | |
|---|---|---|---|---|---|---|---|---|---|
| Age | | | Day 0 | Post-Injection | | Post-Injection | | Post-Injection | |
| (years) | Dog No | Dog Name | Width (mm) | Width (mm) | % Change | Width (mm) | % Change | Width (mm) | % Change |
| 1 | 01RP06 | Dutch | 25 | 26 | 4.0% | 26 | 4.0% | 26 | 4.0% |
| 1 | 09RP05 | Yogui | 22 | 11 | −50.0% | 11 | −50.0% | 11 | −50.0% |
| 1 | 14RH01 | Solovino | 19 | 13 | −31.6% | 13 | −31.6% | 13 | −31.6% |
| 1.5 | 17RM05 | Sultan | 21 | 16 | −23.8% | 16 | −23.8% | 16 | −23.8% |
| 1.5 | 29RP05 | Chicles | 18 | 15 | −16.7% | 15 | −16.7% | 15 | −16.7% |
| 2 | 01RP05 | Pompin | 22 | 15 | −31.8% | 15 | −31.8% | 15 | −31.8% |
| 2 | 03RP05 | Dexter | 27 | 24 | −11.1% | 24 | −11.1% | 24 | −11.1% |
| 2 | 06RP05 | Bobby II | 20 | 10 | −50.0% | 10 | −50.0% | 10 | −50.0% |
| 2 | 10RM05 | Firulais | 27 | 26 | −3.7% | 26 | −3.7% | 26 | −3.7% |
| 2 | 10RM06 | Caruzo | 27 | 14 | −48.1% | 14 | −48.1% | 14 | −48.1% |
| 2 | 11RM06 | Gordo | 27 | 26 | −3.7% | 26 | −3.7% | 26 | −3.7% |
| 2 | 11RP05 | Yanki | 26 | 15 | −42.3% | 15 | −42.3% | 15 | −42.3% |
| 2 | 13RP05A | Rocky | 17 | 12 | −29.4% | 12 | −29.4% | 12 | −29.4% |
| 2 | 13RP05B | Roger | 27 | 26 | −3.7% | 26 | −3.7% | 26 | −3.7% |
| 2 | 15RM05 | Manchas | 21 | 14 | −33.3% | 14 | −33.3% | 14 | −33.3% |
| 2 | 15RP05 | Duque | 27 | 25 | −7.4% | 25 | −7.4% | 25 | −7.4% |
| 2 | 18RP05 | Boby | 21 | 15 | −28.6% | 15 | −28.6% | 15 | −28.6% |
| 2 | 19RM05 | Wester | 26 | 25 | −3.8% | 25 | −3.8% | 25 | −3.8% |
| 2 | 30RP09 | Puppy | 19 | 11 | −42.1% | 11 | −42.1% | 11 | −42.1% |
| 2 | 3RM05 | Killer | 23 | 20 | −13.0% | 20 | −13.0% | 20 | −13.0% |
| 2 | 4RM05 | Rucky | 20 | 20 | 0.0% | 20 | 0.0% | 20 | 0.0% |
| 2 | 4RP06 | Obelix | 27 | 26 | −3.7% | 26 | −3.7% | 26 | −3.7% |
| 2 | 7RM05 | Ramses | 24 | 24 | 0.0% | 24 | 0.0% | 24 | 0.0% |
| 2 | 7RP06 | Ramiro | 24 | 18 | −25.0% | 18 | −25.0% | 18 | −25.0% |
| 2.5 | 1RM06 | Hugo | 27 | 26 | −3.7% | 26 | −3.7% | 26 | −3.7% |
| 3 | 10RH01 | Oso | 16 | 14 | −12.5% | 14 | −12.5% | 14 | −12.5% |
| 3 | 10RP09 | Peluchin | 19 | 14 | −26.3% | 14 | −26.3% | 14 | −26.3% |
| 3 | 14RP09 | Terri | 25 | 25 | 0.0% | 25 | 0.0% | 25 | 0.0% |
| 3 | 15RP09 | Pinto | 24 | 18 | −25.0% | 18 | −25.0% | 18 | −25.0% |
| 3 | 1RH01 | Chacho | 24 | 23 | −4.2% | 23 | −4.2% | 23 | −4.2% |
| 3 | 20RP09 | Tribilin | 16 | 14 | −12.5% | 14 | −12.5% | 14 | −12.5% |
| 3 | 22RM05 | Beethoven | 25 | 11 | −56.0% | 11 | −56.0% | 11 | −56.0% |
| 3 | 2RM06 | Brandy | 27 | 25 | −7.4% | 25 | −7.4% | 25 | −7.4% |
| 3 | 31RP05 | Flay | 22 | 11 | −50.0% | 11 | −50.0% | 11 | −50.0% |
| 3 | 36RP09 | Duque | 23 | 13 | −43.5% | 13 | −43.5% | 13 | −43.5% |
| 3 | 8RM06 | Bloky | 27 | 23 | −14.8% | 23 | −14.8% | 23 | −14.8% |
| 3 | 9RM05 | Bigotes | 21 | 12 | −42.9% | 12 | −42.9% | 12 | −42.9% |
| 4 | 1RH04 | Aragorn | 22 | * | * | * | * | * | * |
| 4 | 26RP05 | Bando | 23 | 16 | −30.4% | 16 | −30.4% | 16 | −30.4% |
| 5 | 11RH01 | Abelardo | 22 | 20 | −9.1% | 20 | −9.1% | 20 | −9.1% |
| 5 | 19RH01 | Tejon | 22 | 16 | −27.3% | 16 | −27.3% | 16 | −27.3% |
| 5 | 2RM05 | Bobby | 27 | 24 | −11.1% | 24 | −11.1% | 24 | −11.1% |
| 5 | 32RP05 | Jordi | 21 | 13 | −38.1% | 13 | −38.1% | 13 | −38.1% |
| 5 | 5RM06 | Peluchin | 17 | 11 | −35.3% | 11 | −35.3% | 11 | −35.3% |
| 5 | 9RM06 | Muneco | 24 | 25 | 4.2% | 25 | 4.2% | 25 | 4.2% |
| 6 | 11RM05 | Scooby | 22 | 15 | −31.8% | 15 | −31.8% | 15 | −31.8% |
| 6 | 13RM05 | Chiquilin I | 20 | 13 | −35.0% | 13 | −35.0% | 13 | −35.0% |
| 6 | 14RM05 | Chiquilin II | 27 | 25 | −7.4% | 25 | −7.4% | 25 | −7.4% |
| 6 | 5RM05 | Bombon | 23 | 14 | −39.1% | 14 | −39.1% | 14 | −39.1% |
| 7 | 13RH01 | Max | 25 | 23 | −8.0% | 23 | −8.0% | 23 | −8.0% |
| 7 | 20RP05 | Golfo | 26 | 18 | −30.8% | 18 | −30.8% | 18 | −30.8% |
| 8 | 18RH01 | Tulipan | 22 | 18 | −18.2% | 18 | −18.2% | 18 | −18.2% |
| 12 | 16RM05 | Blanco | 25 | 11 | −56.0% | 11 | −56.0% | 11 | −56.0% |
| 12 | 6RH01 | Peluche | 19 | 16 | −15.8% | 16 | −15.8% | 16 | −15.8% |

| | | | |
|---|---|---|---|
| * castrated 7 days post-injection | −22.4% | 22.4% | −22.4% |

TABLE IV

| | | | | 2 Months | | 4 Months | | 6 Months | |
|---|---|---|---|---|---|---|---|---|---|
| Age | | | Day 0 | Post-Injection | | Post-Injection | | Post-Injection | |
| (years) | Dog No | Dog Name | Width (mm) | Width (mm) | % Change | Width (mm) | Width (mm) | Width (mm) | % Change |
| 1 | 01RP06 | Dutch | 25 | 26 | 4.0% | 26 | 4.0% | 26 | 4.0% |
| 1 | 09RP05 | Yogui | 22 | 12 | −45.5% | 12 | −45.5% | 12 | −45.5% |
| 1 | 14RH01 | Solovino | 16 | 9 | −43.8% | 9 | −43.8% | 9 | −43.8% |
| 1.5 | 17RM05 | Sultan | 20 | 17 | −15.0% | 17 | −15.0% | 17 | −15.0% |
| 1.5 | 29RP05 | Chicles | 21 | 13 | −38.1% | 13 | −38.1% | 13 | −38.1% |
| 2 | 01RP05 | Pompin | 21 | 11 | −47.6% | 11 | −47.6% | 11 | −47.6% |
| 2 | 03RP05 | Dexter | 26 | 20 | −23.1% | 20 | −23.1% | 20 | −23.1% |
| 2 | 06RP05 | Bobby II | 20 | 10 | −50.0% | 10 | −50.0% | 10 | −50.0% |
| 2 | 10RM05 | Firulais | 27 | 26 | −3.7% | 26 | −3.7% | 26 | −3.7% |
| 2 | 10RM06 | Caruzo | 25 | 16 | −36.0% | 16 | −36.0% | 16 | −36.0% |
| 2 | 11RM06 | Gordo | 27 | 17 | −37.0% | 17 | −37.0% | 17 | −37.0% |
| 2 | 11RP05 | Yanki | 26 | 21 | −19.2% | 21 | −19.2% | 21 | −19.2% |
| 2 | 13RP05A | Rocky | 17 | 9 | −47.1% | 9 | −47.1% | 9 | −47.1% |
| 2 | 13RP05B | Roger | 26 | 25 | −3.8% | 25 | −3.8% | 25 | −3.8% |
| 2 | 15RM05 | Manchas | 23 | 21 | −8.7% | 21 | −8.7% | 21 | −8.7% |
| 2 | 15RP05 | Duque | 27 | 26 | −3.7% | 26 | −3.7% | 26 | −3.7% |
| 2 | 18RP05 | Boby | 24 | 23 | −4.2% | 23 | −4.2% | 23 | −4.2% |
| 2 | 19RM05 | Wester | 25 | 21 | −16.0% | 21 | −16.0% | 21 | −16.0% |
| 2 | 30RP09 | Puppy | 19 | 16 | −15.8% | 16 | −15.8% | 16 | −15.8% |
| 2 | 3RM05 | Killer | 25 | 23 | −8.0% | 23 | −8.0% | 23 | −8.0% |
| 2 | 4RM05 | Rucky | 22 | 21 | −4.5% | 21 | −4.5% | 21 | −4.5% |
| 2 | 4RP06 | Obelix | 27 | 25 | −7.4% | 25 | −7.4% | 25 | −7.4% |
| 2 | 7RM05 | Ramses | 24 | 23 | −4.2% | 23 | −4.2% | 23 | −4.2% |
| 2 | 7RP06 | Ramiro | 25 | 16 | −36.0% | 16 | −36.0% | 16 | −36.0% |
| 2.5 | 1RM06 | Hugo | 27 | 16 | −40.7% | 16 | −40.7% | 16 | −40.7% |
| 3 | 10RH01 | Oso | 16 | 16 | 0.0% | 16 | 0.0% | 16 | 0.0% |
| 3 | 10RP09 | Peluchin | 19 | 15 | −21.1% | 15 | −21.1% | 15 | −21.1% |
| 3 | 14RP09 | Terri | 26 | 21 | −19.2% | 21 | −19.2% | 21 | −19.2% |
| 3 | 15RP09 | Pinto | 24 | 14 | −41.7% | 14 | −41.7% | 14 | −41.7% |
| 3 | 1RH01 | Chacho | 24 | 25 | 4.2% | 25 | 4.2% | 25 | 4.2% |
| 3 | 20RP09 | Tribilin | 23 | 16 | −30.4% | 16 | −30.4% | 16 | −30.4% |
| 3 | 22RM05 | Beethoven | 24 | 11 | −54.2% | 11 | −54.2% | 11 | −54.2% |
| 3 | 2RM06 | Brandy | 27 | 26 | −3.7% | 26 | −3.7% | 26 | −3.7% |
| 3 | 31RP05 | Flay | 24 | 12 | −50.0% | 12 | −50.0% | 12 | −50.0% |
| 3 | 36RP09 | Duque | 25 | 13 | −48.0% | 13 | −48.0% | 13 | −48.0% |
| 3 | 8RM06 | Bloky | 26 | 20 | −23.1% | 20 | −23.1% | 20 | −23.1% |
| 3 | 9RM05 | Bigotes | 24 | 20 | −16.7% | 20 | −16.7% | 20 | −16.7% |
| 4 | 1RH04 | Aragorn | 23 | * | * | * | * | * | * |
| 4 | 26RP05 | Bando | 23 | 14 | −39.1% | 14 | −39.1% | 14 | −39.1% |
| 5 | 11RH01 | Abelarda | 19 | 18 | −5.3% | 18 | −5.3% | 18 | −5.3% |
| 5 | 19RH01 | Tejon | 22 | 15 | −31.8% | 15 | −31.8% | 15 | −31.8% |
| 5 | 2RM05 | Bobby | 24 | 21 | −12.5% | 21 | −12.5% | 21 | −12.5% |
| 5 | 32RP05 | Jordi | 24 | 12 | −50.0% | 12 | −50.0% | 12 | −50.0% |
| 5 | 5RM06 | Peluchin | 19 | 9 | −52.6% | 9 | −52.6% | 9 | −52.6% |
| 5 | 9RM06 | Muneco | 23 | 21 | −8.7% | 21 | −8.7% | 21 | −8.7% |
| 6 | 11RM05 | Scooby | 23 | 18 | −21.7% | 18 | −21.7% | 18 | −21.7% |
| 6 | 13RM05 | Chiquilin I | 21 | 18 | −14.3% | 18 | −14.3% | 18 | −14.3% |
| 6 | 14RM05 | Chiquilin II | 27 | 23 | −14.8% | 23 | −14.8% | 23 | −14.8% |
| 6 | 5RM05 | Bombon | 25 | 12 | −52.0% | 12 | −52.0% | 12 | −52.0% |
| 7 | 13RH01 | Max | 25 | 20 | −20.0% | 20 | −20.0% | 20 | −20.0% |
| 7 | 20RP05 | Golfo | 27 | 12 | −55.6% | 12 | −55.6% | 12 | −55.6% |
| 8 | 18RH01 | Tulipan | 22 | 17 | −22.7% | 17 | −22.7% | 17 | −22.7% |
| 12 | 16RM05 | Blanco | 19 | 13 | −31.6% | 13 | −31.6% | 13 | −31.6% |
| 12 | 6RH01 | Peluche | 19 | 15 | −21.1% | 15 | −21.1% | 15 | −21.1% |

| | | | |
|---|---|---|---|
| * castrated 7 days post-injection | −24.8% | −24.8% | −24.8% |

### EXAMPLE 3

A semen analysis on each of the 54 dogs in Example 1 was done at two months, four months and six months post-injection. Semen analysis was used as the indicator of permanent sterility. In dogs, the spermatogenic cycle is sixty days and, if the animal does not produce viable sperm within two cycles, as documented by semen analysis, then the animal is permanently sterile. The following terms were used relative to semen analysis.

| | | |
|---|---|---|
| (1) | Semen could not | Dog is uncooperative, |
| | be collected: | i.e.temperament, |
| | | untrained in collection |
| (2) | Aspermia: | No semen ejaculated |
| (3) | Azoospermia: | No spermatozoa in the ejaculate |
| (4) | Necrospermia: | Spermatozoa in the ejaculate are |
| | | dead or motionless |
| (5) | Oligospermia: | Less than 20 million spermatozoa |
| | | per mL |

The semen analyses are reported in Table V below.

TABLE V

| | | | Day 0 | | | | | Semen Analysis | | |
|---|---|---|---|---|---|---|---|---|---|---|
| Age | | | Semen | Volume | Motility | Concentration | | 2 Months | 4 Months | 6 Months |
| (years) | Dog No | Dog Name | Analysis | (mL) | (%) | (×10⁶) | Oligospermia* | Post-Injection | Post-Injection | Post-Injection |
| 1 | 01RP06 | Dutch | 5 | 10.0 | 90 | 29.5 | No | 3 | 3 | 3 |
| 1 | 09RP05 | Yogui | 5 | 3.5 | 75 | 59.0 | No | 3 | 3 | 3 |
| 1 | 14RH01 | Solovino | 5 | 5.0 | 95 | 55.5 | No | 3 | 3 | 3 |
| 1.5 | 17RM05 | Sultan | 5 | 1.0 | 20 | 164.5 | No | 3 | 3 | 3 |
| 1.5 | 29RP05 | Chicles | 5 | 2.0 | 90 | 100.0 | No | 3 | 3 | 3 |
| 2 | 01RP05 | Pompin | 5 | 2.5 | 95 | 440.0 | No | 3 | 3 | 3 |
| 2 | 03RP05 | Dexter | 5 | 5.0 | 80 | 450.0 | No | 3 | 3 | 3 |
| 2 | 06RP05 | Bobby II | 5 | 4.5 | 60 | 382.0 | No | 3 | 3 | 3 |
| 2 | 10RM05 | Firulais | 5 | 5.0 | 95 | 150.5 | No | 3 | 3 | 3 |
| 2 | 10RM06 | Caruzo | 5 | 6.0 | 75 | 150.0 | No | 2 | 2 | 2 |
| 2 | 11RM06 | Gordo | 5 | 1.0 | 95 | 1140.0 | No | 3 | 3 | 3 |
| 2 | 11RP05 | Yanki | 5 | 3.5 | 95 | 325.0 | No | 3 | 3 | 3 |
| 2 | 13RP05A | Rocky | 5 | 8.5 | 20 | 122.5 | No | 3 | 3 | 3 |
| 2 | 13RP05B | Roger | 5 | 5.0 | 95 | 199.0 | No | 3 | 3 | 3 |
| 2 | 15RM05 | Manchas | 5 | 9.0 | 95 | 175.0 | No | 3 | 3 | 3 |
| 2 | 15RP05 | Duque | 5 | 3.0 | 80 | 159.5 | No | 3 | 3 | 3 |
| 2 | 18RP05 | Boby | 5 | 3.5 | 50 | 94.5 | No | 3 | 3 | 3 |
| 2 | 19RM05 | Wester | 5 | 2.0 | 20 | 104.0 | No | 3 | 3 | 3 |
| 2 | 30RP09 | Puppy | 5 | 3.5 | 15 | 220.0 | No | 3 | 3 | 3 |
| 2 | 3RM05 | Killer | 5 | 1.0 | 95 | 46.0 | No | 3 | 3 | 3 |
| 2 | 4RM05 | Rucky | 5 | 5.0 | 20 | 96.5 | No | 5 | 5 | 5 |
| 2 | 4RP06 | Obelix | 5 | 5.0 | 85 | 400.0 | No | 3 | 3 | 3 |
| 2 | 7RM05 | Ramses | 5 | 0.5 | 30 | 520.0 | No | 3 | 3 | 3 |
| 2 | 7RP06 | Ramiro | 5 | 9.0 | 95 | 83.5 | No | 3 | 3 | 3 |
| 2.5 | 1RM06 | Hugo | 5 | 11.0 | 70 | 94.5 | No | 3 | 3 | 3 |
| 3 | 10RH01 | Oso | 5 | 8.0 | 60 | 76.0 | No | 3 | 3 | 3 |
| 3 | 10RP09 | Peluchin | 5 | 3.0 | 5 | 170.0 | No | 3 | 3 | 3 |
| 3 | 14RP09 | Terri | 5 | 8.0 | 95 | 102.0 | No | 3 | 3 | 3 |
| 3 | 15RP09 | Pinto | 5 | 6.0 | 80 | 110.0 | No | 3 | 3 | 3 |
| 3 | 1RH01 | Chacho | 5 | 10.0 | 95 | 327.0 | No | 3 | 3 | 3 |
| 3 | 20RP09 | Tribilin | 5 | 5.5 | 60 | 85.5 | No | 3 | 3 | 3 |
| 3 | 22RM05 | Beethoven | 5 | 2.0 | 85 | 316.0 | No | 2 | 2 | 2 |
| 3 | 2RM06 | Brandy | 5 | 13.0 | 95 | 51.0 | No | 2 | 2 | 2 |
| 3 | 31RP05 | Flay | 5 | 15.0 | 90 | 78.5 | No | 3 | 3 | 3 |
| 3 | 36RP09 | Duque | 5 | 5.0 | 60 | 110.0 | No | 3 | 3 | 3 |
| 3 | 8RM06 | Bloky | 5 | 8.0 | 95 | 97.0 | No | 3 | 3 | 3 |
| 3 | 9RM05 | Bigotes | 5 | 5.0 | 95 | 173.5 | No | 3 | 3 | 3 |
| 4 | 1RH04 | Aragorn | 5 | 6.0 | 95 | 656.0 | No | ** | ** | ** |
| 4 | 26RP05 | Bando | 5 | 5.0 | 95 | 115.0 | No | 3 | 3 | 3 |
| 5 | 11RH01 | Abelardo | 5 | 10.0 | 95 | 280.0 | No | 3 | 3 | 3 |
| 5 | 19RH01 | Tejon | 5 | 10.0 | 80 | 126.0 | No | 3 | 3 | 3 |
| 5 | 2RM05 | Bobby | 5 | 11.0 | 95 | 46.0 | No | 3 | 3 | 3 |
| 5 | 32RP05 | Jordi | 5 | 5.0 | 60 | 62.0 | No | 3 | 3 | 3 |
| 5 | 5RM06 | Peluchin | 5 | 2.0 | 85 | 230.0 | No | 2 | 2 | 2 |
| 5 | 9RM06 | Muneco | 5 | 2.0 | 50 | 51.0 | No | 2 | 2 | 2 |
| 6 | 11RM05 | Scooby | 5 | 7.0 | 90 | 120.0 | No | 2 | 2 | 2 |
| 6 | 13RM05 | Chiquilin I | 5 | 8.0 | 95 | 33.0 | No | 2 | 2 | 2 |
| 6 | 14RM05 | Chiquilin II | 5 | 1.0 | 20 | 104.0 | No | 3 | 3 | 3 |
| 6 | 5RM05 | Bombon | 5 | 4.0 | 95 | 613.5 | No | 3 | 2 | 2 |
| 7 | 13RH01 | Max | 5 | 15.0 | 50 | 82.0 | No | 3 | 3 | 3 |
| 7 | 20RP05 | Golfo | 5 | 12.0 | 90 | 55.5 | No | 3 | 3 | 3 |
| 8 | 18RH01 | Tulipan | 5 | 20.0 | 60 | 110.0 | No | 3 | 3 | 3 |
| 12 | 16RM05 | Blanco | 5 | 5.0 | 95 | 70.0 | No | 3 | 3 | 3 |
| 12 | 6RH01 | Peluche | 5 | 7.5 | 95 | 280.0 | No | 2 | 2 | 2 |

5: Sperm in the ejaculate are motile
4: Sperm in ejaculate are dead or motionless (Necrospermia)
3: No sperm in the ejaculate (Azoospermia)
2: No semen ejaculated (Aspermia)
1: Semen could not be collected: dog is uncooperative
* Less than 20 million sperm per mL
** Castrated

In view of the above, it will be seen that the several objects of the invention are achieved and other advantageous results attained. As various changes could be made in the above chemical compositions without departing from the scope of the invention, it is intended that all matter contained in the above description or shown in the accompanying drawings shall be interpreted as illustrative and not in a limiting sense.

What is claimed:

1. The use of a chemical sterilant for adult male dog population control, said chemical sterilant comprising an aqueous solution of a mineral gluconate salt and an amino acid capable of forming the solution, said aqueous solution neutralized to a pH in the range of 6.0 to 7.5 for injection into a sexually mature dog, said sexually mature dog having scrotal testes with seminiferous tubules flowably connected to a head of an epididymis by tubuli recti, rete testis and ductuli efferentes, said sexually mature dog being free-roaming and occupying a position in a hierarchy of free-roaming dogs in a biological niche, said sexually mature dog being captured for sterilization in the biological niche, said sterilant injected into a dorsal cranial portion of each testis beside the head of the epididymis in an effective amount to achieve sterilization without substantially affecting the dog's male physical appearance or secondary sexual characteristics, said sexually mature dog immediately released back into the biological niche.

2. The use of the chemical sterilant according to claim 1 wherein the chemical sterilant is zinc gluconate neutralized with arginine.

3. The use of the chemical sterilant according to claim 2 wherein the zinc gluconate and the arginine are present in substantially equal molar amounts at a concentration in the range from about 0.05 M to about 2.0 M.

4. The use of the chemical sterilant according to claim 3 wherein the concentration of the zinc gluconate and the arginine is from about 0.05 M to about 0.3 M.

5. The use of the chemical sterilant according to claim 4 wherein the concentration of the zinc gluconate and the arginine is from about 0.01 M to about 0.2 M.

6. The use of the chemical sterilant according to claim 5 wherein the chemical sterilant contains 13.1 mg/ml zinc as zinc gluconate and 34.8 mg/ml l-arginine.

7. The use of the chemical sterilant according to claim 6 wherein the sexually mature dog's testes are no less than 10 mm and no more than 27 mm wide and wherein the chemical sterilant is injected in a volume of:
0.2 mL per testis when the testis measures between 10-12 mm,
0.3 mL per testis when the testis measures between 13-15 mm,
0.5 mL per testis when the testis measures between 16-18 mm,
0.7 mL per testis when the testis measures between 19-21 mm,
0.8 mL per testis when the testis measures between 22-24 mm, and
1.0 mL per testis when the testis measures between 25-27 mm.
